(19) United States
(12) Patent Application Publication
(10) Pub. No.: US 2025/0194981 A1
(43) Pub. Date: Jun. 19, 2025

(54) PHYSIOLOGICAL SIGNAL MEASUREMENT SYSTEM, PHYSIOLOGICAL SIGNAL MEASUREMENT METHOD AND PHYSIOLOGICAL SIGNAL MEASUREMENT ELECTRODE

(71) Applicant: INDUSTRIAL TECHNOLOGY RESEARCH INSTITUTE, Hsinchu (TW)

(72) Inventors: Hung-Hsien KO, Zhubei City (TW); Yun-Yi HUANG, Hsinchu City (TW); Chun LIU, Hsinchu City (TW); Yu-Chiao TSAI, Hsinchu City (TW); Heng-Yin CHEN, Hsinchu City (TW)

(73) Assignee: INDUSTRIAL TECHNOLOGY RESEARCH INSTITUTE, Hsinchu (TW)

(21) Appl. No.: 18/607,694

(22) Filed: Mar. 18, 2024

(30) Foreign Application Priority Data
Dec. 19, 2023 (TW) 112149513

(51) Int. Cl. A61B 5/28

(52) U.S. Cl. CPC A61B 5/28

(57) ABSTRACT

A physiological signal measurement system, a physiological signal measurement method and a physiological signal measurement electrode are provided. The physiological signal measurement system includes a first electrode, a second electrode, a plurality of first static electricity receiving electrodes, a plurality of second static electricity receiving electrodes, a transient voltage suppression multiplexer circuit, a signal processing sensor module and a physiological signal analysis module. The first static electricity receiving electrodes are arranged in an array on the first electrode. The second static electricity receiving electrodes are arranged in an array on the second electrode. The transient voltage suppression multiplexer circuit is connected to each of the first static electricity receiving electrodes and each of the second static electricity receiving electrodes. The signal processing sensor module obtains a differential signal according to a first sensing signal and a second sensing signal. The physiological signal analysis module compensates the differential signal.

This application claims the benefit of Taiwan application Serial No. 112149513, filed Dec. 19, 2023, the disclosure of which is incorporated by reference herein in its entirety.

### TECHNICAL FIELD

The disclosure relates to a physiological signal measurement system, a physiological signal measurement method and a physiological signal measurement electrode.

### BACKGROUND

With the continuous advancement of physiological signal measurement technology, many wearable physiological signal measurement devices have been developed. These physiological signal measurement devices could even measure through a fabric or a casing of a portable device.

However, when the physiological signal measurement device is direct contact with the skin, or measurements are made through the fabric or the casing of the portable device, a static electricity often affects the measurement and analysis of the physiological signals.

### SUMMARY

The disclosure is directed to a physiological signal measurement system, a physiological signal measurement method and a physiological signal measurement electrode. The physiological signal measurement electrode designed with a double-layer structure is used to detect electrostatic surges, and then a transient voltage suppression multiplex circuit, a static electricity elimination circuit and a grounding component are used to eliminate the electrostatic surges. Further, a physiological signal analysis module is used to perform the compensation, so that the measured physiological signals could be accurately read.

According to one embodiment, a physiological signal measurement system is provided. The physiological signal measurement system includes a first electrode, a second electrode, a plurality of first static electricity receiving electrodes, a plurality of second static electricity receiving electrodes, at least one transient voltage suppression multiplexer circuit, a signal processing sensor module and a physiological signal analysis module. The first electrode is used for obtaining a first sensing signal. The second electrode is used for obtaining a second sensing signal. The first static electricity receiving electrodes are connected to and arranged in an array on the first electrode. The second static electricity receiving electrodes are connected to and arranged in an array on the second electrode. The transient voltage suppression multiplexer circuit is connected to each of the first static electricity receiving electrodes and each of the second static electricity receiving electrodes, to eliminate a static-electricity surge of the first sensing signal and the second sensing signal. The signal processing sensor module is used for obtaining a differential signal according to the first sensing signal and the second sensing signal. The physiological signal analysis module is used for compensating the differential signal.

According to another embodiment, a physiological signal measurement method is provided. The physiological signal measurement method includes the following steps: obtaining a first sensing signal and a second sensing signal by a first electrode and a second electrode respectively, wherein a plurality of first static electricity receiving electrodes are connected to and arranged in an array on the first electrode, a plurality of second static electricity receiving electrodes are connected to and arranged in an array on the second electrode; eliminating a static-electricity surge of the first sensing signal and the second sensing signal; obtaining a differential signal according to the first sensing signal and the second sensing signal; and compensating the differential signal.

According to an alternative embodiment, a physiological signal measurement electrode is provided. The physiological signal measurement electrode includes a first electrode, a second electrode, a plurality of first static electricity receiving electrodes and a plurality of second static electricity receiving electrodes. The first static electricity receiving electrodes are connected to and arranged in an array on the first electrode. The second static electricity receiving electrodes are connected to and arranged in an array on the second electrode.

### BRIEF DESCRIPTION OF THE DRAWINGS

FIG. 1 illustrates a schematic diagram of measuring a physiological signal according to an embodiment.

FIG. 2 illustrates a block diagram of a physiological signal measurement system according to an embodiment.

FIG. 3 is a schematic diagram of a physiological signal measurement electrode according to an embodiment.

FIG. 4 illustrates a flow chart of a physiological signal measurement method according to an embodiment.

FIG. 5 illustrates a detailed flow chart of the step S140 according to an embodiment.

FIG. 6 illustrates a detailed flow chart of the step S144 according to an embodiment.

FIG. 7 illustrates an example of the implementation of the steps S1441 to S1442 on the EMG signal.

FIG. 8 illustrates an example of the implementation of the step S1443 on the EMG signal.

FIG. 9 illustrates another example of the step S1443 implemented on the EMG signal.

FIG. 10 illustrates another example of step S1443 implemented on the EMG signal.

FIG. 11 illustrates another example of the step S1443 implemented on the EMG signal.

FIG. 12 illustrates an example of the steps S1441 to S1442 being implemented on an ECG signal.

FIG. 13 illustrates an example of the step S1443 being implemented on an ECG signal.

FIG. 14 illustrates a block diagram of a physiological signal measurement system according to another embodiment.

FIG. 15 illustrates a block diagram of a physiological signal measurement system according to another embodiment.

FIG. 16 illustrates a block diagram of a physiological signal measurement system according to another embodiment.

FIG. 17 illustrates a block diagram of a physiological signal measurement system according to another embodiment.

FIG. 18 illustrates a block diagram of a physiological signal measurement system according to another embodiment.

FIG. 19 illustrates a block diagram of a physiological signal measurement system according to another embodiment.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

### DETAILED DESCRIPTION

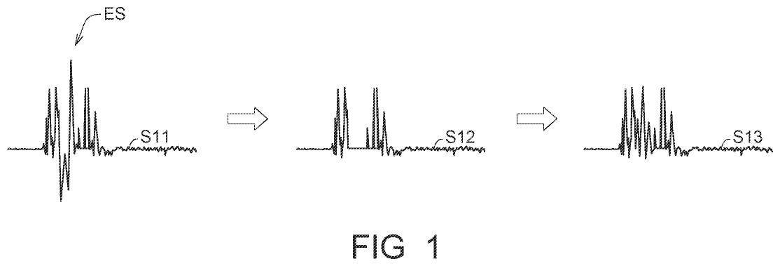

Please refer to FIG. 1, which illustrates a schematic diagram of measuring a physiological signal according to an embodiment. When an Electrocardiography (ECG) signal or an Electromyography (EMG) signal is measured by directly contacting the skin or contacting through a fabric or a casing of a portable device, a physiological signal S11 including a static-electricity surge ES may be obtained.

In this disclosure, after the static-electricity surge ES on the physiological signal S11 is detected, the static-electricity surge ES could be eliminated to obtain a physiological signal S12 without the static-electricity surge ES.

After eliminating the static-electricity surge ES, the physiological signal S12 becomes unreal; so, in this disclosure, the compensation is further processed on the physiological signal S12 to obtain a compensated physiological signal S13. The compensated physiological signal S13 has no static-electricity surge ES and is close to the real physiological signal content, so it could greatly increase the accuracy of signal analysis.

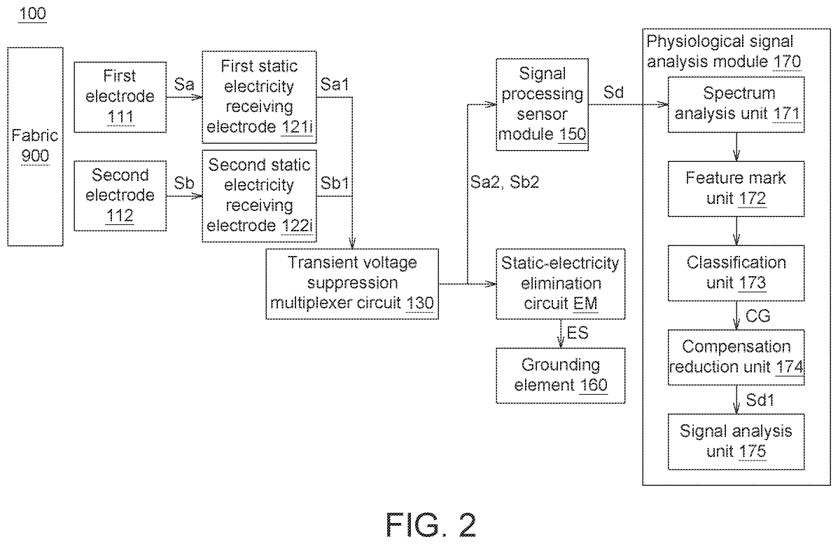

Please refer to FIG. 2, which illustrates a block diagram of a physiological signal measurement system 100 according to an embodiment. The physiological signal measurement system 100 includes a first electrode 111, a second electrode 112, a plurality of first static electricity receiving electrodes 121i, a plurality of second static electricity receiving electrodes 122i, at least one transient voltage suppression multiplexer circuit 130, a signal processing sensor module 150, a static-electricity elimination circuit EM, a grounding element 160 and a physiological signal analysis module 170. The first electrode 111 and the first static electricity receiving electrodes 121i are used to obtain a first sensing signal Sa. The second electrode 112 and the second static electricity receiving electrodes 122i are used to obtain a second sensing signal Sb. After the first sensing signal Sa is inputted to the first static electricity receiving electrodes 121i, a first sensing signal Sa1 is outputted. After the second sensing signal Sb is inputted to the second static electricity receiving electrodes 122i, a second sensing signal Sb1 is outputted. After the first sensing signal Sa1 and the second sensing signal Sb1 are inputted to the transient voltage suppression multiplexer circuit 130, a first sensing signal Sa2 and a second sensing signal Sb2 are outputted. The transient voltage suppression multiplexer circuit 130, the static-electricity elimination circuit EM and the grounding element 160 are used to eliminate the static-electricity surge ES on the first sensing signal Sa2 and the second sensing signal Sb2. The signal processing sensor module 150 is used to obtain a differential signal Sd according to the first sensing signal Sa2 and the second sensing signal Sb2. The physiological signal analysis module 170 is used to compensate the differential signal Sd to obtain a differential signal Sd1. The signal processing sensor module 150 and/or the physiological signal analysis module 170 is, for example, a circuit, a chip, a circuit board or a storage device that stores program code. In this embodiment, the first electrode 111, the second electrode 112, the first static electricity receiving electrodes 121i and the second static electricity receiving electrodes 122i adopt a double-layer structure design to successfully detect the static-electricity surge ES. The following is a detailed description of the double-layer structure design of the first electrode 111, the second electrode 112, the first static electricity receiving electrodes 121i and the second static electricity receiving electrodes 122i.

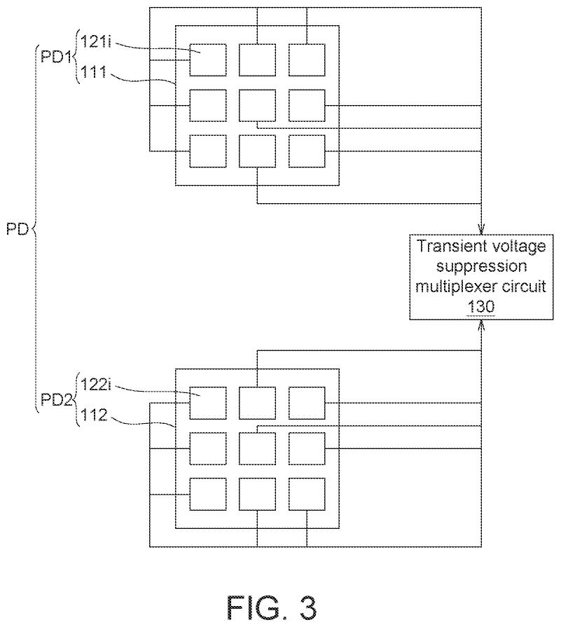

Please refer to FIGS. 2 and 3. FIG. 3 is a schematic diagram of a physiological signal measurement electrode PD according to an embodiment. The first electrode 111, the second electrode 112, the first static electricity receiving electrodes 121i and the second static electricity receiving electrodes 122i form the physiological signal measurement electrode PD with the double-layer structure design. The first electrode 111 is used to contact a fabric 900 (or the skin). The fabric 900 is shown in FIG. 2. The first static electricity receiving electrodes 121i are connected and arranged in an array on the first electrode 111, and form a first sensing pad PD1. The first electrode 111 is located between the fabric 900 (or the skin) and the first static electricity receiving electrodes 121i. The second electrode 112 is used to contact the fabric 900 (or the skin). The second static electricity receiving electrodes 122i are connected and arranged in an array on the second electrode 112, and form a second sensing pad PD2. The second electrode 112 is located between the fabric 900 (or the skin) and the second static electricity receiving electrodes 122i. The first electrode 111 receives the first sensing signal Sa of the subject through the fabric 900 (or directly in contact with the skin). The second electrode 112 receives the second sensing signal Sb of the subject through the fabric 900 (or directly in contact with the skin).

In the first sensing pad PD1, each of the first static electricity receiving electrodes 121i is smaller than the first electrode 111. For example, the first static electricity receiving electrodes 121i have substantially the same sizes. In the second sensing pad PD2, each of the second static electricity receiving electrodes 122i is smaller than the second electrode 112. For example, the second static electricity receiving electrodes 122i have substantially the same sizes. In some embodiments, the size of the first sensing pad PD1 or the second sensing pad PD2 may be quite large, for example, covering the entire upper arm or the entire palm. Therefore, the first sensing pad PD1 and the second sensing pad PD2 may generate different degrees of static-electricity surge ES at different times and positions. The first static electricity receiving electrodes 121i and the second static electricity receiving electrodes 122i arranged in an array could receive the static-electricity surge ES quickly and instantly at various locations to avoid interference in the signal measurement caused by accumulation of the static electricity.

In this embodiment, the static-electricity surge ES could be smoothly eliminated through the design of the transient voltage suppression multiplexer circuit 130, the static-electricity elimination circuit EM and the grounding element 160, so as to avoid the static-electricity surge ES from interfering with the interpretation for the first sensing signal Sa2 and the second sensing signal Sb2. The following is a flow chart that explains the operation of each component in detail.

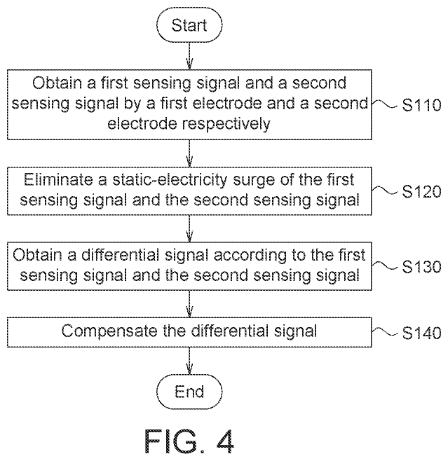

Please refer to FIG. 2 and FIG. 4 at the same time. FIG. 4 illustrates a flow chart of a physiological signal measurement method according to an embodiment. The physiological signal measurement method includes steps S110 to S140. In the step S110, the first sensing signal Sa and the second sensing signal Sb are obtained by the first electrode 111 and the second electrode 112 respectively. During the process of collecting the first sensing signal Sa and the second sensing signal Sb, the first static electricity receiving electrodes 121i and the second static electricity receiving electrodes 122i may receive the static-electricity surge ES at various locations. The static-electricity surge ES at different locations will be received and transmitted by the corresponding first static electricity receiving electrodes 121i and/or the corresponding second static electricity receiving electrodes 122i. As long as the static-electricity surge ES occurs somewhere, the static-electricity surge ES could be immediately transmitted to the transient voltage suppression multiplexer circuit 130 by the first static electricity receiving electrodes 121i and the second static electricity receiving electrodes 122i.

Then, in the step S120, the transient voltage suppression multiplexer circuit 130 and the static-electricity elimination circuit EM eliminate the static-electricity surge ES on the first sensing signal Sa2 and the second sensing signal Sb2. When the static-electricity surge ES is transmitted to the transient voltage suppression multiplexer circuit 130, the transient voltage suppression multiplexer circuit 130 will immediately activate the static-electricity elimination circuit EM, so that the static-electricity surge ES is directed to the grounding element 160. The static-electricity elimination circuit EM does not always direct the signal to the grounding element 160, but when there is the static-electricity surge ES, the signal will be directed to the grounding element 160. During the grounding, not only the static-electricity surge ES will be eliminated, but also part of the physiological signal.

Then, in the step S130, the signal processing sensor module 150 obtains a differential signal Sd according to the first sensing signal Sa2 and the second sensing signal Sb2. Based on the aforementioned elimination of the static-electricity surge ES, part of the content of the first sensing signal Sa2 and the second sensing signal Sb2 will be eliminated, so part of the content of the differential signal Sd will also be eliminated.

Then, entering the step S140, the physiological signal analysis module 170 compensates the differential signal Sd to obtain the differential signal Sd1. Since part of the content of the differential signal Sd is eliminated, the differential signal Sd would be compensated in this step.

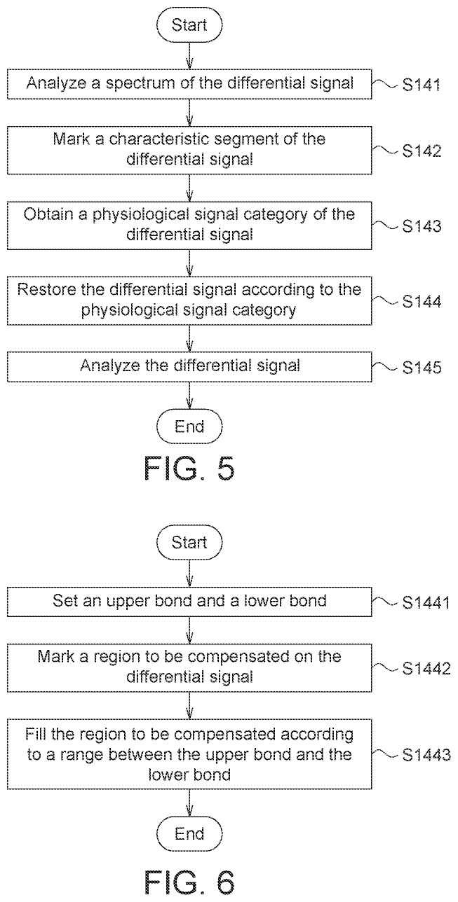

Please refer to FIGS. 2 and 5. FIG. 5 illustrates a detailed flow chart of the step S140 according to an embodiment. As shown in FIG. 2, the physiological signal analysis module 170 includes a spectrum analysis unit 171, a feature mark unit 172, a classification unit 173, a compensation reduction unit 174 and a signal analysis unit 175. The spectrum analysis unit 171, the feature mark unit 172, the classification unit 173, the compensation reduction unit 174 and the signal analysis unit 175 are used to perform various analysis and processing procedures for the differential signal Sd. The detailed flow chart in the FIG. 5 illustrates the operation of each component.

As shown in the FIG. 5, the step S140 includes steps S141 to S145. In the step S141, the spectrum analysis unit 171 of the physiological signal analysis module 170 analyzes the spectrum of the differential signal Sd.

Next, in the step S142, the feature mark unit 172 of the physiological signal analysis module 170 marks a feature segment of the differential signal Sd.

Then, in the step S143, the classification unit 173 of the physiological signal analysis module 170 obtains a physiological signal category CG of the differential signal Sd. For example, the differential signal Sd is classified into an ECG signal or an EMG signal. The different categories of the physiological signal category CG will be performed different processing procedures at the subsequent steps.

Afterwards, in the step S144, the compensation reduction unit 174 of the physiological signal analysis module 170 restores the differential signal Sd to the differential signal Sd1 according to the physiological signal category CG. In this step, the cleared content of the physiological signal could be restored according to the physiological signal category CG.

Then, in the step S145, the signal analysis unit 175 of the physiological signal analysis module 170 analyzes the differential signal Sd1 for subsequent recording and analysis of the physiological data.

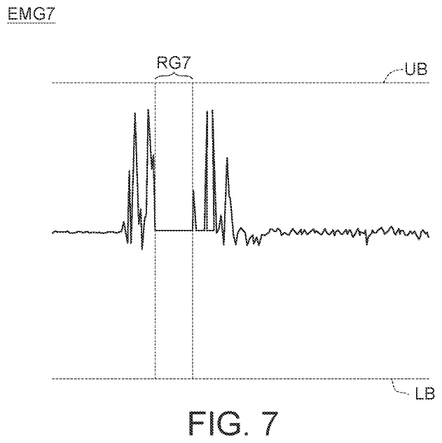

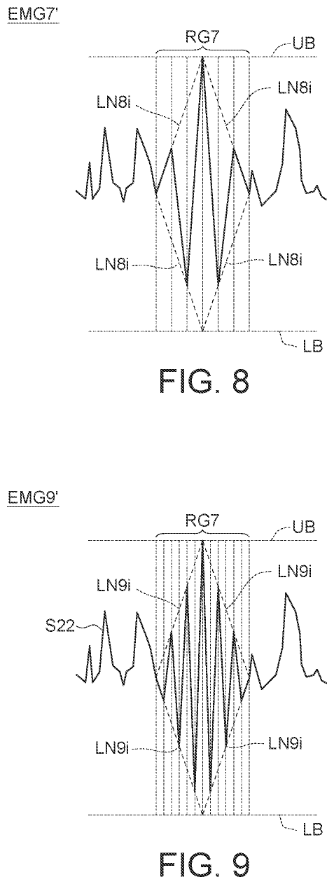

Please refer to FIGS. 6 to 8. FIG. 6 illustrates a detailed flow chart of the step S144 according to an embodiment. FIG. 7 illustrates an example of the implementation of the steps S1441 to S1442 on the EMG signal. FIG. 8 illustrates an example of the implementation of the step S1443 on the EMG signal. The step S144 in the FIG. 5 above could be further subdivided into steps S1441 to S1443 in FIG. 6.

In the step S1441, as shown in FIG. 7, the compensation reduction unit 174 of the physiological signal analysis module 170 sets an upper bond UB and a lower bond LB. The upper bond UB and the lower bond LB are the maximum possible value and the smallest possible value of the conventional EMG signal.

Next, in the step S1442, as shown in FIG. 7, the compensation reduction unit 174 of the physiological signal analysis module 170 marks a region RG7 to be compensated on the differential signal EMG7. The region RG7 to be compensated is the range of the physiological signal that is cleared.

Then, in the step S1443, as shown in FIG. 8, the compensation reduction unit 174 of the physiological signal analysis module 170 fills the region RG7 to be compensated within the range of the upper bond UB and the lower bond LB. In this step, the compensation reduction unit 174, for example, fills the region RG7 to be compensated in a linear manner.

For example, the compensation reduction unit 174 draws four symmetrical straight lines LN8i in the region RG7 to be compensated within the range of the upper bond UB and the lower bond LB. The compensation reduction unit 174 divides the region RG7 to be compensated into 6 equal parts, and then marks several peaks on the straight line LN8i to obtain a restored differential signal EMG7′. In the example shown in the FIG. 8, the compensation reduction unit 174 fills the region RG7 to be compensated with multiple symmetrical peaks.

Please refer to FIG. 9, which illustrates another example of the step S1443 implemented on the EMG signal. In this embodiment, the compensation reduction unit 174 draws four symmetrical straight lines LN9i in the region RG7 to be compensated within the range of the upper bond UB and the lower bond LB. The compensation reduction unit 174 divides the region RG7 to be compensated RG7 into 12 equal parts, and then marks several peaks on the straight line LN9i to obtain a restored differential signal EMG9′. In the example shown in the FIG. 9, the compensation reduction unit 174 fills the region RG7 to be compensated with multiple symmetrical wave peaks.

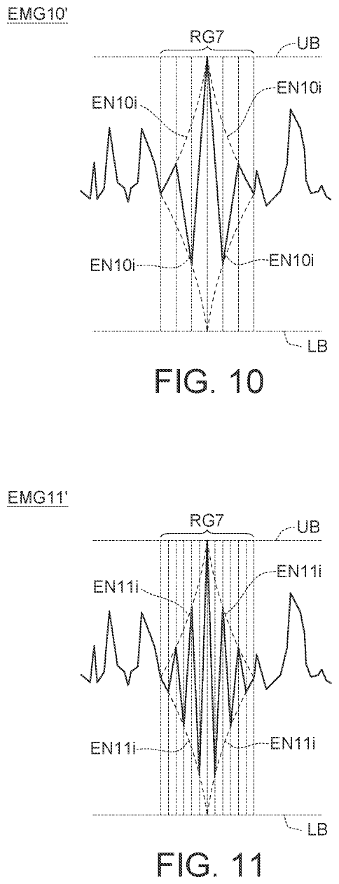

Please refer to FIG. 10, which illustrates another example of step S1443 implemented on the EMG signal. In this embodiment, the compensation reduction unit 174 draws four symmetrical exponential curves EN10i in the region RG7 to be compensated within the range of the upper bond UB and the lower bond LB. The compensation reduction unit 174 divides the region RG7 to be compensated into 6 equal parts, and then marks several peaks on the exponential curve EN10i to obtain a restored differential signal EMG10′. In the example shown in the FIG. 10, the compensation reduction unit 174 fills the region RG7 to be compensated with multiple symmetrical wave peaks.

Please refer to FIG. 11, which illustrates another example of the step S1443 implemented on the EMG signal. In this embodiment, the compensation reduction unit 174 draws four symmetrical exponential curves EN11i in the region RG7 to be compensated within the range of the upper bond UB and the lower bond LB. The compensation reduction unit 174 divides the region RG7 to be compensated into 12 equal parts, and then marks several peaks on the exponential curve EN11i to obtain a restored differential signal EMG11′. In the example shown in the FIG. 11, the compensation reduction unit 174 fills the region RG7 to be compensated with multiple symmetrical wave peaks.

According to the above embodiments in FIGS. 6 to 11, the EMG signal after eliminating the static-electricity surge ES could be smoothly compensated in a linear or nonlinear manner to retrieve the true content of the physiological signal.

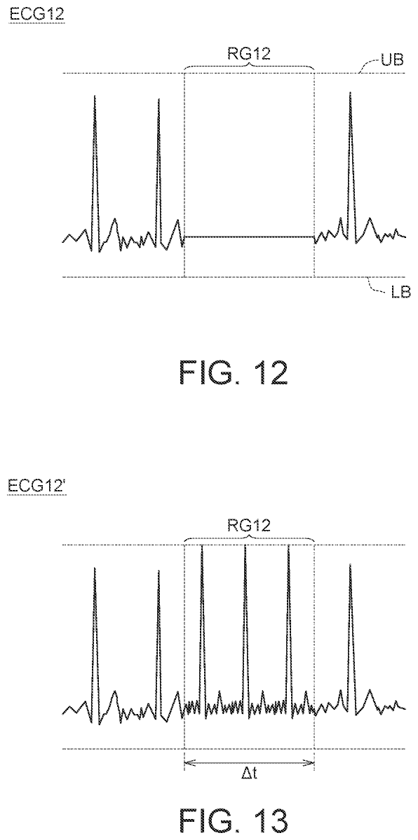

Please refer to FIG. 12 and FIG. 13. FIG. 12 illustrates an example of the steps S1441 to S1442 being implemented on an ECG signal. FIG. 13 illustrates an example of the step S1443 being implemented on an ECG signal. Taking the ECG signal as an example, in the step S1441, as shown in FIG. 12, the compensation reduction unit 174 of the physiological signal analysis module 170 sets the upper bond UB and the lower bond LB.

Next, in the step S1442, as shown in FIG. 12, the compensation reduction unit 174 of the physiological signal analysis module 170 marks a region RG12 to be compensated on the differential signal ECG12.

Then, in the step S1443, as shown in FIG. 13, the compensation reduction unit 174 of the physiological signal analysis module 170 fills the region RG12 to be compensated within the range of the upper bond UB and the lower bond LB. In this step, the compensation reduction unit 174 analyzes the length of time Δt in the region RG12 to be compensated, and analyzes the number of ECG waveforms occupied by the length of time Δt. Based on this number, the compensation reduction unit 174 fills one or more ECG waveforms in the region RG12 to be compensated to obtain a compensated differential signal ECG12′.

According to the above embodiments in FIGS. 12 to 13, the ECG signal after eliminating the static-electricity surge ES could be smoothly compensated through insertion to retrieve the true physiological signal content.

In the above embodiment, the static-electricity elimination circuit EM is disposed between the transient voltage suppression multiplexer circuit 130 and the grounding element 160. In another embodiment, the static-electricity elimination circuit EM could be disposed at other locations.

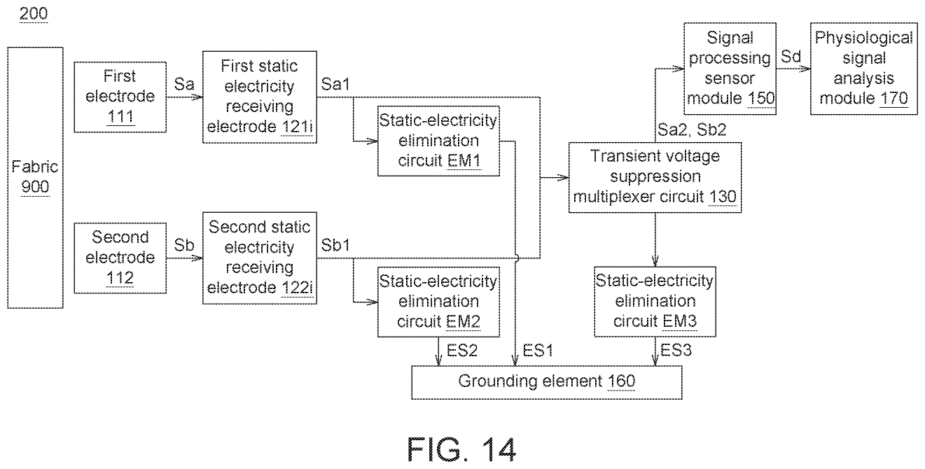

Please refer to FIG. 14, which illustrates a block diagram of a physiological signal measurement system 200 according to another embodiment. In this embodiment, the physiological signal measurement system 200 includes three static-electricity elimination circuits EM1, EM2, EM3. The three static-electricity elimination circuits EM1, EM2, EM3 are respectively connected to the first static electricity receiving electrodes 121i, the second static electricity receiving electrodes 122i and the transient voltage suppression multiplexer circuit 130. The static-electricity elimination circuits EM1, EM2, EM3 are connected to the grounding element 160 for grounding. The static-electricity surges ES1, ES2, ES3 are excluded respectively. In this embodiment, the first static electricity receiving electrodes 121i are directly connected to the static-electricity elimination circuit EM1, so once each position of the first static electricity receiving electrodes 121i is interfered by the static electricity, the static-electricity surge ES1 could be instantly eliminated. Similarly, the second static electricity receiving electrodes 122i are directly connected to the static-electricity elimination circuit EM2, so once each position of the second static electricity receiving electrodes 122i is interfered by the static electricity, the static-electricity surge ES2 could also be eliminated immediately. The static-electricity elimination circuit EM3 connected to the transient voltage suppression multiplexer circuit 130 could detect and eliminate the static-electricity surge ES3 on the first sensing signal Sa1 and the second sensing signal Sb1.

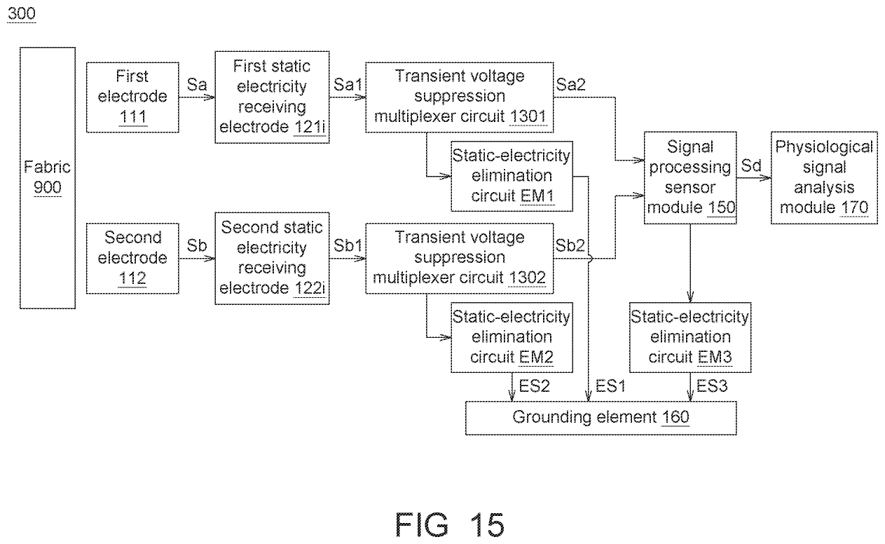

Please refer to FIG. 15, which illustrates a block diagram of a physiological signal measurement system 300 according to another embodiment. In this embodiment, the physiological signal measurement system 300 includes two transient voltage suppression multiplexer circuits 1301, 1302 and three static-electricity elimination circuits EM1, EM2, EM3. The two transient voltage suppression multiplexer circuits 1301, 1302 are respectively connected to the first static electricity receiving electrodes 121i and the second static electricity receiving electrodes 122i. The three static-electricity elimination circuits EM1, EM2, EM3 are respectively connected to the two transient voltage suppression multiplexer circuits 1301, 1302 and the signal processing sensor module 150. The static-electricity elimination circuits EM1, EM2, EM3 are connected to the grounding element 160 for grounding to eliminate the static-electricity surges ES1, ES2, ES3 respectively. In this embodiment, the transient voltage suppression multiplexer circuits 1301 and 1302 are connected to the static-electricity elimination circuits EM1 and EM2 respectively. Therefore, the transient voltage suppression multiplexer circuit 1301 and the static-electricity elimination circuit EM1 could independently detect and eliminate the electricity surge ES1 on the entire first sensing signal Sa2. The transient voltage suppression multiplexer circuit 1302 and the static-electricity elimination circuit EM2 could independently detect and eliminate the static-electricity surge ES2 on the entire second sensing signal Sb2. In addition, the static-electricity elimination circuit EM3 connected to the signal processing sensor module 150 could independently detect and eliminate the static-electricity surge ES3 on the differential signal Sd.

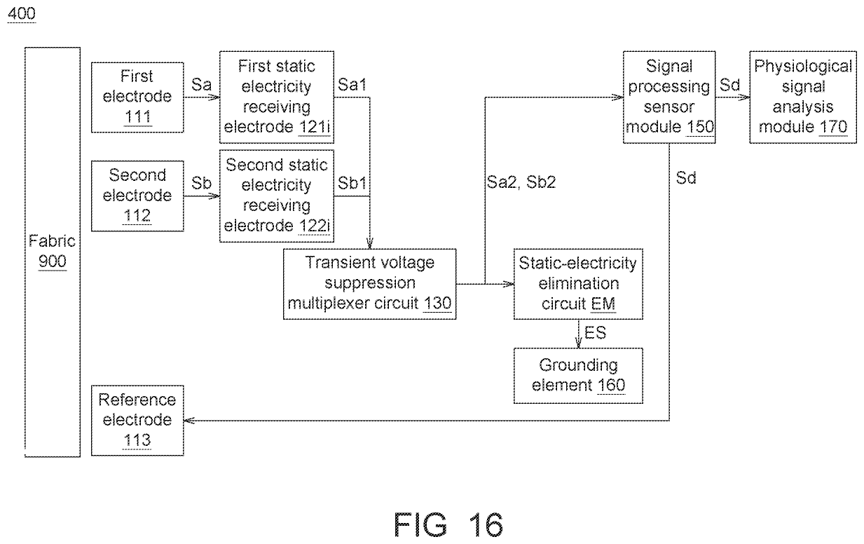

Please refer to FIG. 16, which illustrates a block diagram of a physiological signal measurement system 400 according to another embodiment. In this embodiment, the physiological signal measurement system 400 further includes a reference electrode 113. The signal processing sensor module 150 is connected to the reference electrode 113. When the signal processing sensor module 150 obtains the differential signal Sd according to the first sensing signal Sa2 and the second sensing signal Sb2, the differential signal Sd could be fed back to the reference electrode 113.

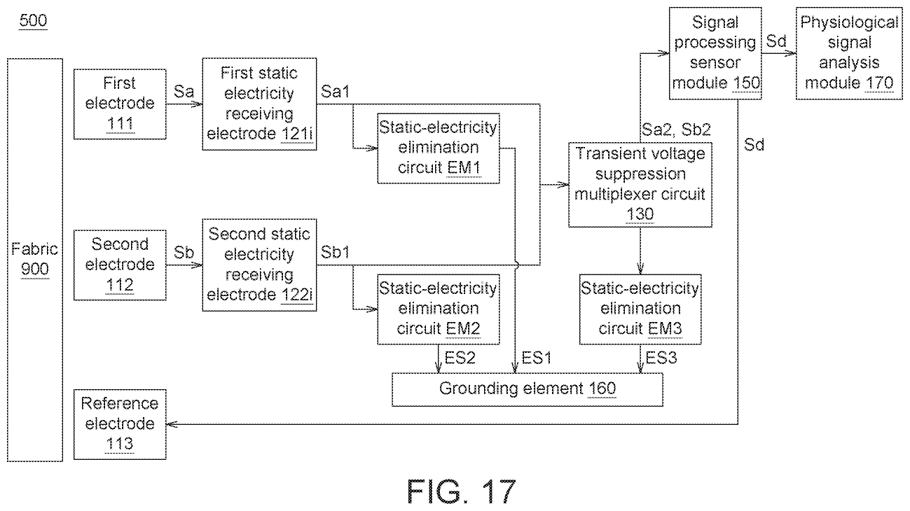

Please refer to FIG. 17, which illustrates a block diagram of a physiological signal measurement system 500 according to another embodiment. The physiological signal measurement system 500 includes three static-electricity elimination circuits EM1, EM2, EM3 and a reference electrode 113. The three static-electricity elimination circuits EM1, EM2, EM3 are respectively connected to the first static electricity receiving electrodes 121i, the second static electricity receiving electrodes 122i and the transient voltage suppression multiplexer circuit 130. The static-electricity elimination circuits EM1, EM2, EM3 are connected to the grounding element 160 for grounding. The static-electricity surges ES1, ES2, ES3 are excluded respectively. In this embodiment, the first static electricity receiving electrodes 121i are directly connected to the static-electricity elimination circuit EM1, so once each position of the first static electricity receiving electrodes 121i is interfered by the static electricity, the static-electricity surge ES1 could be instantly eliminated. Similarly, the second static electricity receiving electrodes 122i are directly connected to the static-electricity elimination circuit EM2, so once each position of the second static electricity receiving electrodes 122i are interfered by the static electricity, the static-electricity surge ES2 could also be eliminated immediately. The static-electricity elimination circuit EM3 connected to the transient voltage suppression multiplexer circuit 130 could detect and eliminate the static-electricity surge ES3 on the first sensing signal Sa2 and the second sensing signal Sb2.

The signal processing sensor module 150 is connected to the reference electrode 113. When the signal processing sensor module 150 obtains the differential signal Sd according to the first sensing signal Sa2 and the second sensing signal Sb2, the differential signal Sd could be fed back to the reference electrode 113.

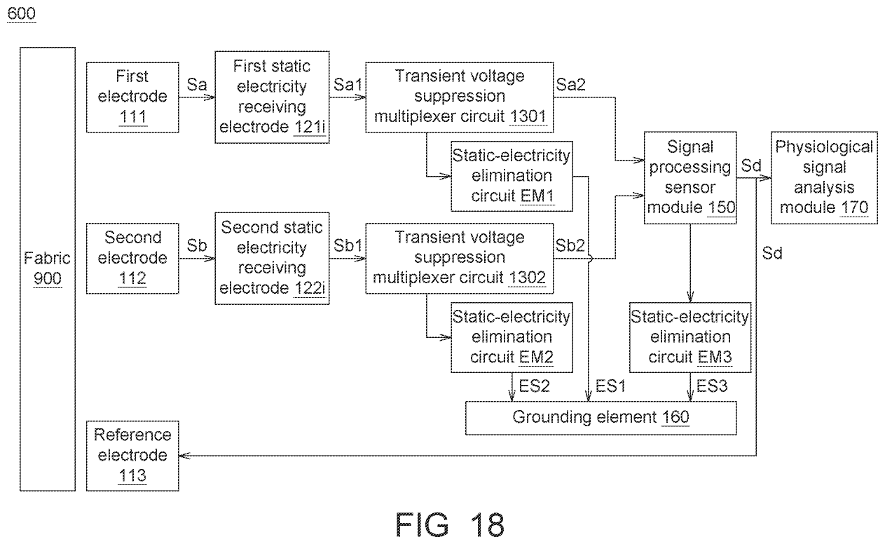

Please refer to FIG. 18, which illustrates a block diagram of a physiological signal measurement system 600 according to another embodiment. In this embodiment, the physiological signal measurement system 600 includes two transient voltage suppression multiplexer circuits 1301, 1302, three static-electricity elimination circuits EM1, EM2, EM3, and a reference electrode 113. The two transient voltage suppression multiplexer circuits 1301, 1302 are respectively connected to the first static electricity receiving electrodes 121i and the second static electricity receiving electrodes 122i. The three static-electricity elimination circuits EM1, EM2, EM3 are respectively connected to the two transient voltage suppression multiplexer circuits 1301, 1302 and the signal processing sensor module 150. The static-electricity elimination circuits EM1, EM2, EM3 are connected to the grounding element 160 for grounding to eliminate the static-electricity surges ES1, ES2, ES3 respectively. In this embodiment, the transient voltage suppression multiplexer circuits 1301, 1302 are connected to the static-electricity elimination circuits EM1, EM2 respectively. The transient voltage suppression multiplexer circuit 1301 and the static-electricity elimination circuit EM1 could independently detect and eliminate the static-electricity surge ES1 on the first sensing signal Sa2. The transient voltage suppression multiplexer circuit 1302 and the static-electricity elimination circuit EM2 could independently detect and eliminate the static-electricity surge ES2 on the second sensing signal Sb2. In addition, the static-electricity elimination circuit EM3 connected to the signal processing sensor module 150 could independently detect and eliminate the static-electricity surge ES3 on the differential signal Sd.

The signal processing sensor module 150 is connected to the reference electrode 113. When the signal processing sensor module 150 obtains the differential signal Sd according to the first sensing signal Sa2 and the second sensing signal Sb2, the differential signal Sd could be fed back to the reference electrode 113.

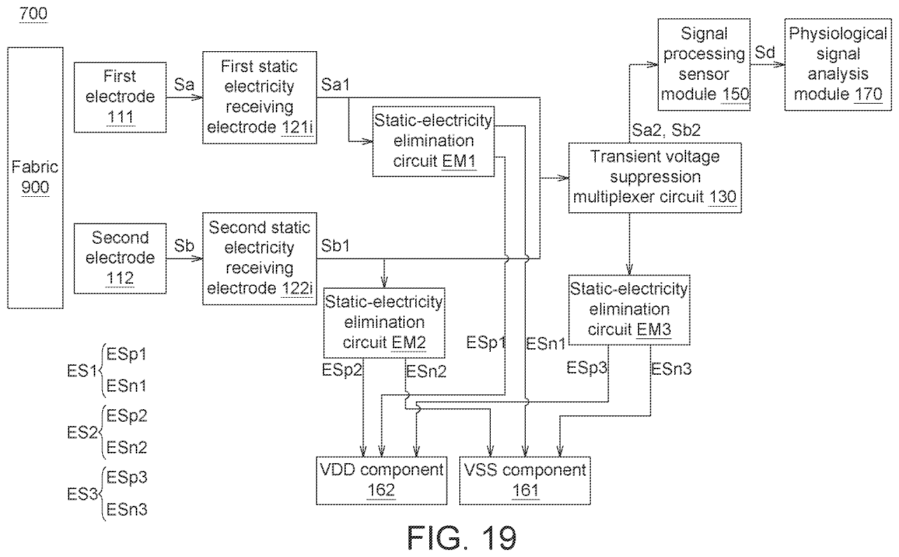

Please refer to FIG. 19, which illustrates a block diagram of a physiological signal measurement system 700 according to another embodiment. In this embodiment, the physiological signal measurement system 700 includes three static-electricity elimination circuits EM1, EM2, EM3, a VSS component 161 and a VDD component 162. The three static-electricity elimination circuits EM1, EM2, EM3 are respectively connected to the first static electricity receiving electrodes 121i, the second static electricity receiving electrodes 122i and the transient voltage suppression multiplexer circuit 130. The static-electricity elimination circuits EM1, EM2, EM3 are connected to the VSS component 161 and the VDD component 162. In this embodiment, the first static electricity receiving electrodes 121i are directly connected to the static-electricity elimination circuit EM1, so once each position of the first static electricity receiving electrodes 121i is interfered by the static electricity, the static-electricity surge ES1 could be eliminated immediately. Similarly, the second static electricity receiving electrodes 122i are directly connected to the static-electricity elimination circuit EM2, so once each position of the second static electricity receiving electrodes 122i is interfered by the static electricity, the static-electricity surge ES2 could also be eliminated immediately. The static-electricity elimination circuit EM3 connected to the transient voltage suppression multiplexer circuit 130 could detect and eliminate the static-electricity surge ES3 on the first sensing signal Sa2 and the second sensing signal Sb2.

In this embodiment, the negative voltage parts ESn1, ESn2, ESn3 of the static-electricity surge ES1, ES2, ES3 could be directed to the VSS component 161; the positive voltage parts ESp1, ESp2, ESp3 of the static-electricity surge ES1, ES2, ES3, could be directed to the VDD component 162.

According to the above embodiments, the first electrode 111, the second electrode 112, the first static electricity receiving electrodes 121i and the second static electricity receiving electrodes 122i adopted the double-layer structure design could successfully detect the static-electricity surge ES, ES1, ES2, ES3. Through the design of the transient voltage suppression multiplexer circuit 130, 1301, 1302, the static-electricity elimination circuit EM, EM1, EM2, EM3, the grounding element 160, the VSS component 161, the VDD component 162, the static-electricity surge ES, ES1, ES2, ES3 could be smoothly eliminated. Moreover, through the design of the physiological signal analysis module 170, the signal could be compensated, so that the first sensing signal Sa and the second sensing signal Sb could be accurately interpreted.

It will be apparent to those skilled in the art that various modifications and variations could be made to the disclosed embodiments. It is intended that the specification and examples be considered as exemplars only, with a true scope of the disclosure being indicated by the following claims and their equivalents.

What is claimed is:

1. A physiological signal measurement system, comprising:
a first electrode, used for obtaining a first sensing signal;
a second electrode, used for obtaining a second sensing signal;
a plurality of first static electricity receiving electrodes, connected to and arranged in an array on the first electrode;
a plurality of second static electricity receiving electrodes, connected to and arranged in an array on the second electrode;
at least one transient voltage suppression multiplexer circuit, connected to each of the first static electricity receiving electrodes and each of the second static electricity receiving electrodes, to eliminate a static-electricity surge of the first sensing signal and the second sensing signal;
a signal processing sensor module, used for obtaining a differential signal according to the first sensing signal and the second sensing signal; and
a physiological signal analysis module, used for compensating the differential signal.

2. The physiological signal measurement system according to claim 1, further comprising:
a static-electricity elimination circuit, connected to the transient voltage suppression multiplexer circuit, wherein when the static-electricity surge is transmitted to the transient voltage suppression multiplexer circuit, the transient voltage suppression multiplexer circuit activates the static-electricity elimination circuit, to eliminate the static-electricity surge of the first sensing signal and the second sensing signal.

3. The physiological signal measurement system according to claim 1, wherein the physiological signal analysis module comprises:
a spectrum analysis unit, used for analyzing a spectrum of the differential signal;
a feature mark unit, used for marking a characteristic segment of the differential signal;
a classification unit, used for obtaining a physiological signal category of the differential signal;
a compensation reduction unit, used for restoring the differential signal according to the physiological signal category; and
a signal analysis unit, used for analyzing the differential signal.

4. The physiological signal measurement system according to claim 3, wherein the compensation reduction unit is used to set an upper bond and a lower bond, and mark a region to be compensated on the differential signal, and the compensation reduction unit fills the region to be compensated according to a range between the upper bond and the lower bond.

5. The physiological signal measurement system according to claim 4, wherein the compensation reduction unit fills the region to be compensated in a linear manner.

6. The physiological signal measurement system according to claim 4, wherein the compensation reduction unit fills the region to be compensated in an exponential manner.

7. The physiological signal measurement system according to claim 4, wherein the compensation reduction unit fills the region to be compensated in a symmetrical manner.

8. The physiological signal measurement system according to claim 4, wherein the compensation reduction unit fills the region to be compensated with multiple peaks.

9. A physiological signal measurement method, comprising:
obtaining a first sensing signal and a second sensing signal by a first electrode and a second electrode respectively, wherein a plurality of first static electricity receiving electrodes are connected to and arranged in an array on the first electrode, a plurality of second static electricity receiving electrodes are connected to and arranged in an array on the second electrode;
eliminating a static-electricity surge of the first sensing signal and the second sensing signal;
obtaining a differential signal according to the first sensing signal and the second sensing signal; and
compensating the differential signal.

10. The physiological signal measurement method according to claim 9, wherein compensating the differential signal includes:
analyzing a spectrum of the differential signal;
marking a characteristic segment of the differential signal;
obtaining a physiological signal category of the differential signal;
restoring the differential signal according to the physiological signal category; and
analyzing the differential signal.

11. The physiological signal measurement method according to claim 10, wherein restoring the differential signal according to the physiological signal category includes:
setting an upper bond and a lower bond;
marking a region to be compensated on the differential signal; and
filling the region to be compensated according to a range between the upper bond and the lower bond.

12. The physiological signal measurement method according to claim 11, wherein the region to be compensated is filled in a linear manner.

13. The physiological signal measurement method according to claim 11, wherein the region to be compensated is filled in an exponential manner.

14. The physiological signal measurement method according to claim 11, wherein the region to be compensated is filled in a symmetrical manner.

15. The physiological signal measurement method according to claim 11, wherein the region to be compensated is filled with multiple peaks.

16. A physiological signal measurement electrode, comprising:
a first electrode;
a second electrode;
a plurality of first static electricity receiving electrodes, connected to and arranged in an array on the first electrode; and
a plurality of second static electricity receiving electrodes, connected to and arranged in an array on the second electrode.

17. The physiological signal measurement electrode according to claim 16, wherein each of the first static electricity receiving electrodes is smaller than the first electrode, and each of the second static electricity receiving electrodes is smaller than the second electrode.

18. The physiological signal measurement electrode according to claim 16, wherein the first static electricity receiving electrodes have substantially same sizes, and the second static electricity receiving electrodes have substantially same sizes.

19. The physiological signal measurement electrode according to claim 16, wherein the first electrode and the second electrode are used to contact a fabric or a skin, the first electrode is located between the fabric and the first static electricity receiving electrodes or located between the skin and the first static electricity receiving electrodes, and the second electrode is located between the fabric and the second static electricity receiving electrodes or located between the skin and the second static electricity receiving electrodes.
